(19) United States
(12) Patent Application Publication
(10) Pub. No.: US 2024/0221518 A1
(43) Pub. Date: Jul. 4, 2024

(54) SYSTEM AND METHOD FOR VIRTUAL ONLINE MEDICAL TEAM TRAINING AND ASSESSMENT

(71) Applicant: Abdul Karim Qayumi, West Vancouver (CA)

(72) Inventor: Abdul Karim Qayumi, West Vancouver (CA)

(21) Appl. No.: 17/496,726

(22) Filed: Oct. 7, 2021

Related U.S. Application Data
(60) Provisional application No. 63/088,992, filed on Oct. 7, 2020.

(51) Int. Cl. G09B 5/06; G06Q 10/0639; G16H 40/20

(52) U.S. Cl. CPC G09B 5/065; G06Q 10/06398; G16H 40/20

(57) ABSTRACT

There is disclosed a system and method for training and providing an assessment of medical Teams. In an embodiment, the system comprises: a virtual simulated hospital environment having a plurality of simulated objects; and a virtual Patient Avatar configured to provide various Team training scenarios; whereby, a plurality of Team Members individually represented by Team Member Avatars interact with the Patient Avatar and the plurality of simulated objects to perform one or more Team training scenarios on the Patient Avatar within the virtual simulated hospital environment. In another embodiment, the Team training is conducted through one or more of audio and video interaction and animation between the Team Members, and with one or more Instructors. In another embodiment, the Team training is conducted through one or more of haptic feedback interactions between the Team Members and the Patient Avatar.

### CROSS-REFERENCE TO RELATED APPLICATIONS

This application claims the benefit of U.S. Provisional Patent Application No. 63/088,992 filed on Oct. 7, 2020, which is incorporated by reference herein in its entirety.

### FIELD OF THE INVENTION

The present disclosure relates generally to medical Team training and assessment.

### BACKGROUND

One of the gaps identified in medical education is individual training in a professional health education environment and Team performance in real life in a clinical environment. This gap has proven to increase the cost of healthcare, and an increase in complications and medical errors that may lead to increase morbidity and mortality in healthcare systems. Therefore, Team training has become one of the most important aspects of medical education. Learning communication and other soft skills prior to the patient encounter has saved many lives, and reduced morbidity and mortality in critically sick patients. At this time, the only way effective Team training can be provided is with the use of a mannequin simulator in a simulation center, where the physical environment is created specifically for Team training. While effective, mannequin Team training has significant problems and obstacles, including the requirement of a high-fidelity mannequin with its purchase cost and high maintenance and operating costs. Conventional Team training further requires a costly physical space, equipment, instruments, and numerous accessories such as cameras and recording systems to provide a simulation environment, extra space for debriefing, and more. Furthermore, given space constraints, only one group of students can use the space and equipment at a time. This may also inconvenience people who have to travel to the location and limit the participants to Team Members from a local geographic area.

Therefore, what is needed is an alternative solution for medical Team training which addresses at least some of these limitations in the prior art.

### SUMMARY

The present disclosure relates generally to a system and method for virtual online medical Team training.

In an aspect, there is disclosed an online platform for medical Team training which provides a digital platform and digital objects or tools to support and provide a virtual clinical environment for Team training and assessment.

In an embodiment, the system and method provides a virtual training environment for an emergency/trauma bay which reflects a realistic 3D Emergency Room, functional bed, Emergency Room (ER) equipment (such as a ventilator, ECG machine, defibrillator and others), accessories (such as intubation equipment, syringes, IV polls, medication and others), monitoring equipment, video recording, and audio communication sharing capabilities.

In another embodiment, Members of the Team are represented by their Team Member Avatars in the virtual room. Every Team Member will have their Team Member Avatar in the ER and the Team Members are in full control of their respective Team Member Avatars. Team Members—through their Team Member Avatars—are able to perform specific tasks in the environment and on the Patient Avatar.

In an embodiment, Team Member Avatars are generated by Team Members to resemble themselves. These Team Member Avatars will be controlled by individual Team Members within the virtual environment. Their movement, actions and speech will be regulated by Team Members. They will be capable of doing anything that a doctor, nurse or health professional that controls them can do. In this instance, avatars will be able to talk to each other, to the leader of the Team, and to the Patient Avatar. Therefore, the system will function like real-life and there is no need for a separate audio system link in this version to be controlled by the Instructor

In another embodiment, a Patient Avatar serves as the subject of manipulation by Members of the Team. Patient Avatar can be created by Team Members (student/teacher) with specific characteristics such as age, gender, ethnicity etc. The Patient Avatar will be programmed with specific health conditions based on preconceived pathological conditions and when the Avatar is manipulated by Team Members, further development of the case can follow through distinct conditions:

In another environment, an Instructor provides active control of the learning environment. In this environment the condition of the Patient Avatar is fully controlled by the Instructor. The actions are controlled by trainers however the consequences of the actions and further direction of the case is determined by the Instructor. For example, if the learners are making a wrong decision for a medication or delay the process, the Instructor will be able to make the condition of the Patient Avatar worse and vice versa. In this case, the teachers monitor the action of the learners and are able to direct further development of the case and make changes on the status of the Patient Avatar based on the learner's actions. The Instructors are experts in the field, and they can guide the consequences their actions based on the clinical experience of the Instructor/teacher. This will provide the conditions for the Team to make their next decision and perform the tasks that will lead to further consequences. The Instructor is able to change parameters of the Patient Avatar accordingly as desired by the learning objectives.

In another environment, the system and method provide the controls and an Instructor remains passive. In this instance the Instructor's role is passive and without the ability to control the Patient Avatar's condition. The Patient Avatar is controlled automatically based on an algorithm, machine learning and artificial intelligence. In this case, all physiological parameters including vital signs, lab tests and others will be automatically changed based on decisions made and tasks performed on the Patient Avatar. Physiological parameters, vital signs and other conditions of the Patient Avatar change according to the time passed after the task is performed, the type and amount of medication given to the patient and may go into a variety of levels of improvement or deterioration of the Patient Avatar's condition. The Team has to adjust their decisions and action plans as the patient's health condition progresses. In this case, all variations for action, interaction and consequences of action will be analyzed and algorithms will be designed for each of those scenarios. The Machine Learning and Artificial Intelligence will have the capability to choose an appropriate algorithm based on thousands of variations of the parameters that can possibly change due to learner's treatment.

Advantageously, the system and method eliminates at least some of the above described limitations, including requiring substantially less manpower, cost, and elimination of a requirement of an expensive high-fidelity mannequin. Furthermore, given that it is a virtual online space, the need for a dedicated space, equipment, and staff to operate and maintain the equipment is eliminated, and allows students to repeat and practice as many times as needed. Furthermore, the Members of the medical Team could be from anywhere, and an unlimited number of sessions can be running at the same time with unique programmed cases and assessments. An automatized version of system and method can also be used for self-directed studies and practice without the need of an instructor.

In this respect, before explaining at least one embodiment of the invention in detail, it is to be understood that the invention is not limited in its application to the details of construction and to the arrangements of the components set forth in the following description or the examples provided therein, or illustrated in the drawings. Therefore, it will be appreciated that a number of variants and modifications can be made without departing from the teachings of the disclosure as a whole. Therefore, the present system, method and apparatus is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

The present system and method will be better understood, and objects of the invention will become apparent, when consideration is given to the following detailed description thereof. Such description makes reference to the annexed drawings, wherein:

FIG. 1 shows a schematic block diagram of a system and method in a pre-simulation phase accordance with an illustrative embodiment.

FIG. 2 shows a schematic block diagram of a system and method in a simulation phase in accordance with an illustrative embodiment.

FIG. 3 shows a schematic block diagram of system and method in a post-simulation phase in accordance with an illustrative embodiment.

FIG. 4 shows a patient ER decision tree in accordance with an illustrative embodiment.

FIG. 5 shows a patient ER flow in accordance with an illustrative embodiment.

FIG. 6A shows an illustrative Team Member/leader first-person point of view in accordance with an illustrative embodiment.

FIG. 6B shows an illustrative user interface selection screen for selecting user interface parameters in accordance with an illustrative embodiment.

FIGS. 7A-9A show illustrative user interface screens of the present system in accordance with illustrative embodiments.

FIG. 9B shows an illustrative front page, and FIG. 10A shows an illustrative splash page of the present system in accordance with an illustrative embodiment.

FIG. 10B shows an illustrative login page in accordance with an illustrative embodiment.

FIG. 11A shows a simulation room selection page in accordance with an illustrative embodiment.

FIG. 11B shows a pop-up notification window in accordance with an illustrative embodiment.

FIG. 12A shows a role selection page in accordance with an illustrative embodiment.

FIG. 12B shows a simulation screen of a Patient Avatar in accordance with an illustrative embodiment.

FIG. 13A shows a simulation screen with a participating Team Member in accordance with an illustrative embodiment.

FIG. 13B shows an on boarding pop-up page for a participating Team Member in accordance with an illustrative embodiment.

FIGS. 14A and 14B show an on-call waiting and debriefing room in accordance with an illustrative embodiment.

FIGS. 15A-17D show illustrative simulation screens with various objects with which participating Team Members may interact.

FIGS. 18A-18E show illustrative user interface screens for selecting and customizing participating Team Member Avatars in accordance with an illustrative embodiment.

FIG. 19 shows a schematic block diagram of a generic computer which may provide a platform for various embodiments of the present system and method.

In the drawings, embodiments are illustrated by way of example. It is to be expressly understood that the description and drawings are only for the purpose of illustration and as an aid to understanding and are not intended as describing the accurate performance and behavior of the embodiments and a definition of the limits of the invention.

### DETAILED DESCRIPTION

As noted above, the present disclosure relates to a system and method for virtual online medical Team training.

In an aspect, there is disclosed an online platform for medical Team training which provides a digital platform/tool to support and provide a virtual clinical environment for Team training and assessment.

In an embodiment, the system and method provide a virtual training environment for an emergency/trauma bay. This will reflect a realistic 3D Emergency Room, functional bed, ER equipment (such as a ventilator, ECG machine, defibrillator and others), accessories (such as intubation equipment, syringes, IV polls, medication and others), monitoring equipment, video recording, and audio communication sharing capabilities.

In another embodiment, Members of the Team are represented by their Team Member Avatars in the virtual room. Every Team Member will have their Team Member Avatar in the ER and the Team Members are in full control of their respective Team Member Avatars. Team Members—through their Team Member Avatars—are able to perform specific tasks in the environment and on the Patient Avatar.

In another embodiment, a Patient Avatar serves as the subject of manipulation by Members of the Team. A Patient Avatar can be created by Team Members (student/teacher) with specific characteristics such as age, gender, ethnicity etc. The Patient Avatar will be programmed with specific health conditions based on preconceived pathological conditions and when the Avatar is manipulated by Team Members, further development of the case can follow through distinct conditions:

In another environment, an Instructor provides active control of the learning environment. In this condition the simulation environment is fully controlled by the Instructor. The actions are controlled by trainers however the consequences of the actions and further direction of the case is determined by the Instructor. For example, if the learners are making a wrong decision for a medication or delay the process, the Instructor will be able to make the condition of the Patient Avatar worse and vice versa. In this case, the teachers monitor the action of the learners and are able to direct further development of the case and make changes on the status of the Patient Avatar based on the learner's actions. The Instructors are experts in the field, and they can guide the consequences their actions based on the clinical experience of the Instructor/teacher. This will provide the conditions for the Team to make their next decision and perform the tasks that will lead to further consequences. The Instructor is able to change parameters of the Patient Avatar accordingly as desired by the learning objectives.

In another environment, the system and method provide the controls and an Instructor remains passive. In this instance the Instructor's role is passive and without the ability to control the Patient Avatar's condition. The Patient Avatar is controlled automatically based on an algorithm, machine learning and artificial intelligence. In this case, all physiological parameters including vital signs, lab tests and others will be automatically changed based on decisions made and tasks performed on the Patient Avatar. Physiological parameters, vital signs and other conditions of the Patient Avatar change according to the time passed after the task is performed, the type and amount of medication given to the patient and may go into a variety of levels of improvement or deterioration of the Patient Avatar's condition. The Team has to adjust their decisions and action plans as the patient's health condition progresses. In this case, all variations for action, interaction and consequences of action will be analyzed and algorithms will be designed for each of those scenarios. The Machine Learning and Artificial Intelligence will have the capability to choose an appropriate algorithm based on thousands of variations of the parameters that can possibly change due to learner's treatment.

In another embodiment, the Patient Avatar is be generated automatically by learners or Instructors. A Patient Avatar may be in any age, sex, race, or geographical specifications, and will have all normal physiological and anatomical parameters. The user will be able to customize the Patient Avatar into any pathology they choose to simulate. These avatars will also be equipped and programmed with all possible algorithms and artificial intelligence to respond to environmental changes such as drugs, oxygen, temperature and others. They will also respond to actions imposed on them by Members of the Team. In other words, the Patient Avatar will act like a human being when they are affected by drugs (i.e. they will have a dose-dependent response on the effect of a drug and their physiological parameters will change those dependently to mimic the effect of the drug.)

Advantageously, the system and method eliminate at least some of the above described limitations, including requiring substantially less manpower, cost, and elimination of a requirement of an expensive high-fidelity mannequin. Furthermore, given that it is a virtual online space, the need for a dedicated space, equipment, and staff to operate and maintain the equipment is eliminated, and allows students to repeat and practice as many times as needed. Furthermore, the Members of the medical Team could be from anywhere, and an unlimited number of sessions can be running at the same time with unique programmed cases and assessments.

Various illustrative embodiments will now be described with reference to the figures.

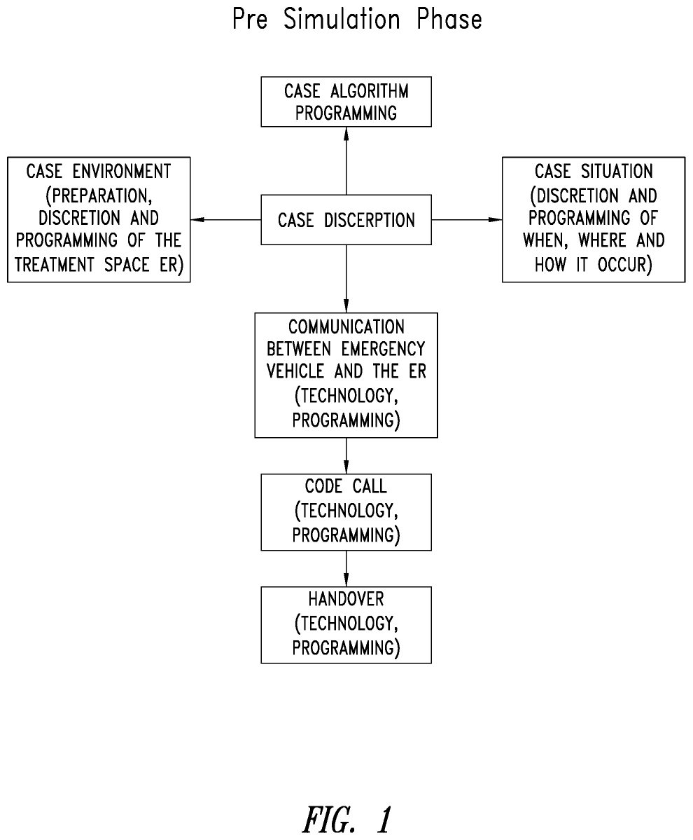

FIG. 1 shows a schematic block diagram of a system and method in a pre-simulation phase accordance with an illustrative embodiment. As illustrated, in the pre-simulation phase, attention is made to prepare the case and the environment for the simulation of a patient case. This requires a significant amount of knowledge preparation, algorithm discerption and programming. It also requires programming for online object interaction and multipoint commutation technology adaptation.

In a simulation pre-simulation phase, Team dynamic is the center of attention. Communication between the Team leader and the other Members of the Team will be assessed by an Instructor or AI. Preferably, the session will be videotaped with time lapse to emphasize the desirable or undesirable actions in the debriefing time. Each time when the Team performance completes and communication loop is closed between the Team and the leader, a new condition imposed to the Team (by the Instructor or the machine) that would require a new action plan and performance from the Team. This cycle that may lead to improvement or deterioration can be repeated many times up until the final decision is made.

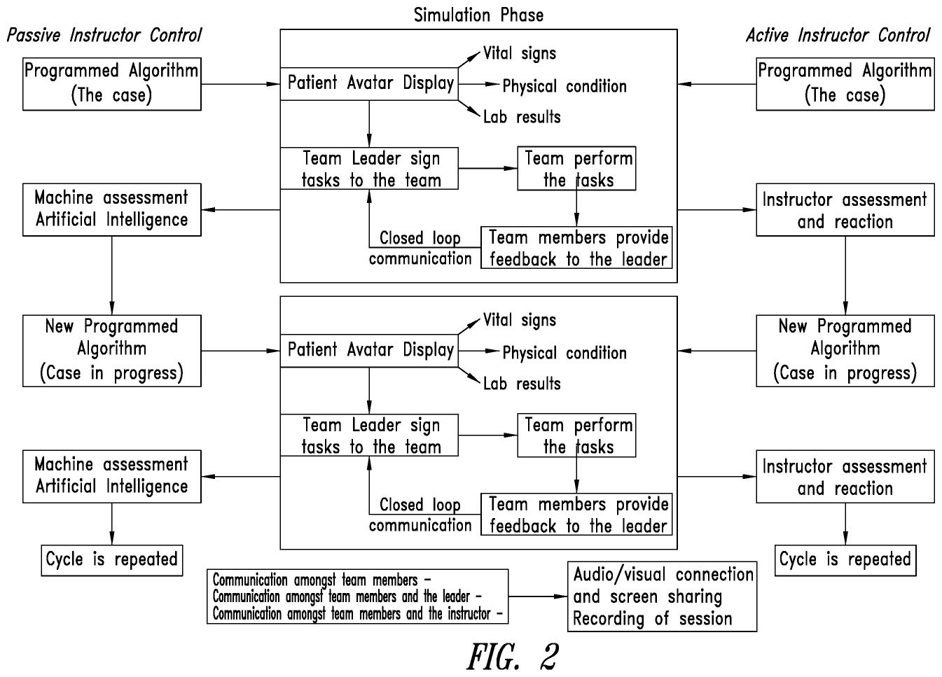

Now referring to FIG. 2, shown is a schematic block diagram of a system and method in a simulation phase in accordance with an illustrative embodiment. In this simulation phase, Team dynamic is the center of attention. Communication between the Team leader and the other Members of the Team will be assessed by an Instructor or AI. The session will be videotaped with time lapse to emphasize the desirable or undesirable actions in the debriefing time. Each time when the Team performance completes and communication loop is closed between the Team and the leader, a new condition imposed to the Team (by the Instructor or the machine) that would require a new action plan and performance from the Team. This cycle that may lead to improvement or deterioration can be repeated many times up until the final decision is made.

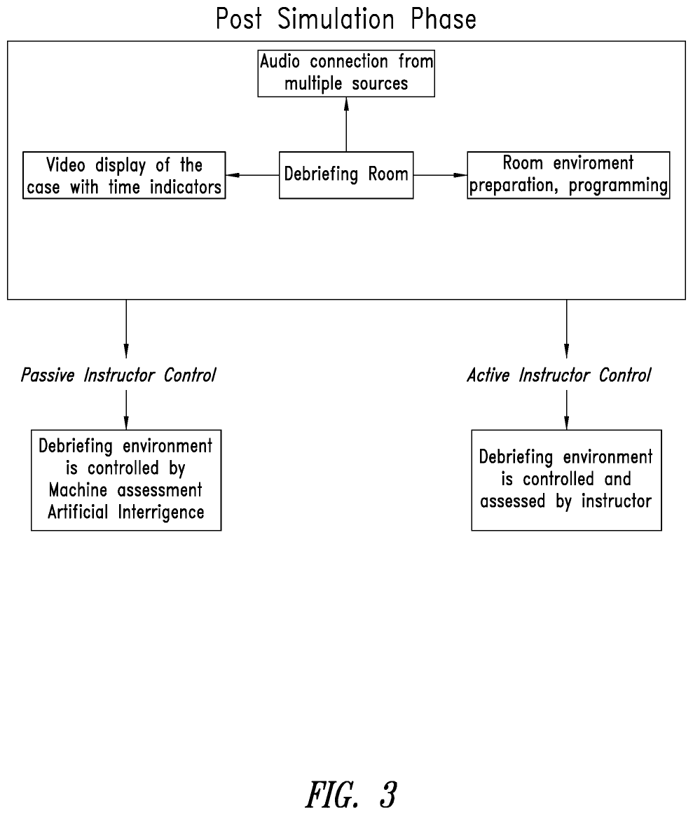

Now referring to FIG. 3, shown is a schematic block diagram of system and method in a post-simulation phase in accordance with an illustrative embodiment. This post simulation phase includes a debriefing and provides the most pedagogical value for participating student Team Members. From the technology point of view requires multipoint communication system, transfer of the time-relapse audiovisual clips from the simulation session to the room and display of the event.

In an embodiment, the system may be viewed as a multi-person online simulation. The simulation is a virtual imitation of a real emergency situation training that some medical students/professionals may experience. The virtual room consists of medical assets, a mannequin, avatars and voice chat. In this simulation, medical professionals and students are able to communicate live in real-time. The users are able to play the simulation from anywhere as long as they have a steady internet connection. The simulated emergency room world has no dangers, special moments or rewards for the Team Members. The challenges in the virtual space are defined by the scenario the facilitator chooses to play through. The Team can then effectively communicate with each other (Closed Loop Communication) to solve the challenge. The user interface of the simulation helps the Team Member interact with the world so that it mimics similar experiences in real life.

For participating Team Members, as noted above, the whole experience in this application consists of three stages: pre-simulation, simulation and debrief (post-simulation). The visuals in the pre-simulation stage are flat designs with a three-dimensional (3D) feeling. The assets in the simulation stage are three-dimensional recreations of the emergency room in real life.

In terms of sound, in the pre-simulation stage, a scenario is described or presented verbally, and may be utilized for the assignment of duties to the team members. However, when choosing a virtual room to the end of the simulation, users are able to activate the voice chat function to verbally communicate with other Team Members. Once they enter the simulation room, the scene will be recorded. In the simulation stage, the “beep” sound of the vital machine and phone ringing sound are implemented. The vital machine sound recreates the environment in the real emergency room and gives Team Members a sense of emergency. The phone ringing sound signifies that a report has arrived in the virtual emergency room.

After users finish the simulation, the facilitator leads them to a debrief session where they will review the recorded video about what they have done in the simulation. The debrief session consists of the recorded video of the simulation as well as time bookmarks made by the facilitator. The overall look of the debrief session is similar to a video conference where the instructor and all team members can see each other talk to each other and watch the video of their performance.

In an embodiment, the system provides a user journey map which sketches out a user experience, mirror user interactions, and forecast any friction before creating the functional prototypes. It included users' needs, actions, thoughts, feelings, emotions, and a potential route to reach a particular goal in the simulation. The system defines the user journey for the persona as the following:

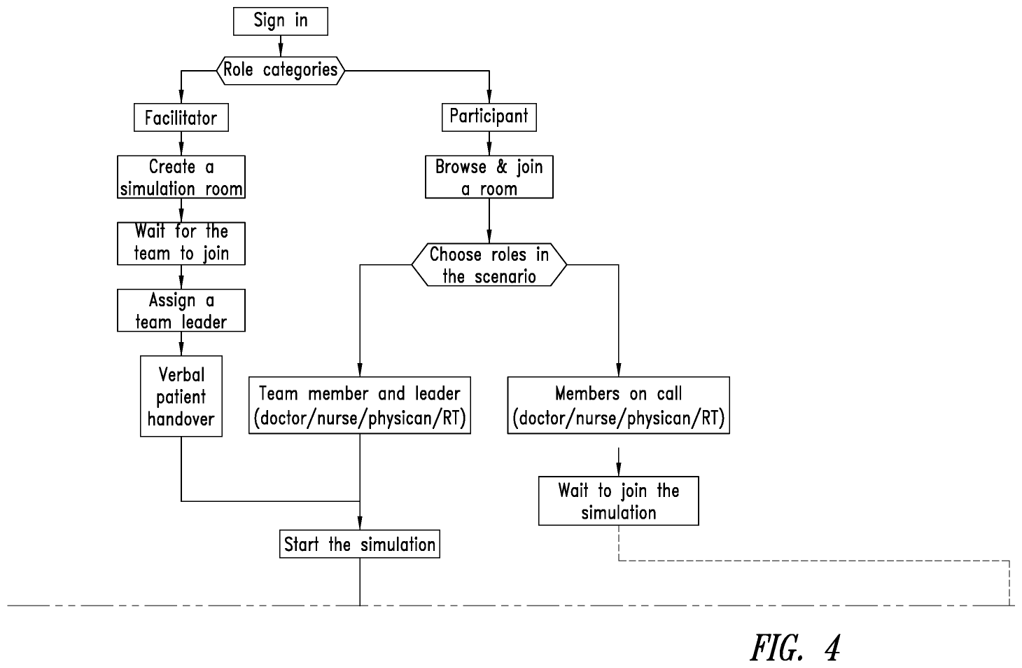

Now referring to FIG. 4, shown is a patient decision tree in accordance with an illustrative embodiment. In the present system, users sign in as the “facilitator” or “participant.” The whole Team can be joined in a pre-simulation area or in the simulation room for selection of each Member's role. The facilitator may then initiate the patient handover and start the simulation when the Team is ready.

For the Team, once they sign in and choose the room, they need to wait for the Team leader to assign roles through the voice chat function. After they enter the room, their avatars appear in their spawn positions (around the patient bed), they can interact with the mannequin and the environment based on the tasks they receive from the Team leader. For the Team leader, his/her avatar usually stands at the end of the patient bed, and he/she gives tasks to each Member via voice chat. On some occasions, the Team leader can participate in the tasks with Team Members.

The interfaces of the Team and the facilitator are different at the simulation phase. During the simulation in the virtual ER, the facilitator's screen is mainly used as a control panel. He/she is responsible for the monitoring of Team performance. He/she can mark a time point if there is something that he/she wants to note down. The facilitator can also change the mannequin's vitals (blood pressure, heart rate, pulse, respiratory rate, and oxygen saturation) and mimic mannequin's sounds (moaning). When the facilitator notices that the Team requests medical reports (ECG, x-ray, blood test), he/she needs to choose the right report and send it to the Team.

Once the Team finishes the tasks in the virtual simulation room, the facilitator clicks “end simulation” and the whole Team goes to the debrief session.

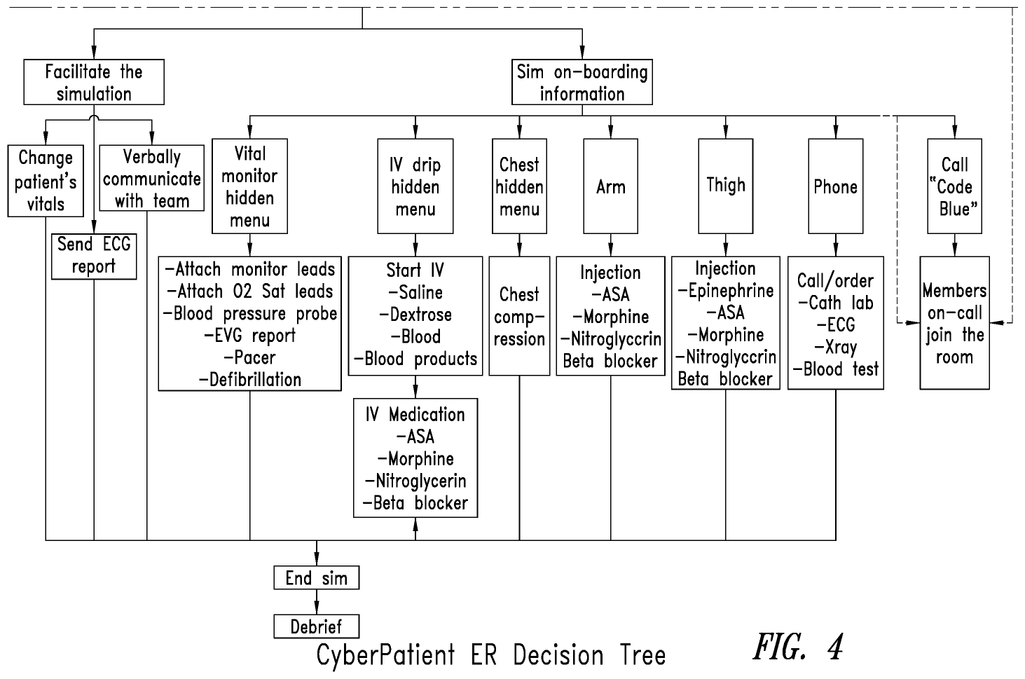

Now referring to FIG. 5, shown is a patient flow in accordance with an illustrative embodiment. The flow chart in FIG. 5 shows what users are expected to see and do during their whole experience in the prototype.

During the pre-simulation and debrief session (as well as during the simulation for the facilitator) users interact with the application without their avatars. Upon entering the virtual emergency room, each user has an avatar as the embodiment of themselves in the virtual emergency room. Team Members' see through the first-person point of view, which means that the camera is positioned to the avatar's forehead looking forward. The user sees what the avatar sees. This can lead to a greater sense of connection with the avatar and a higher sense of immersion. From each user's viewpoint, he/she can see what other avatars are doing.

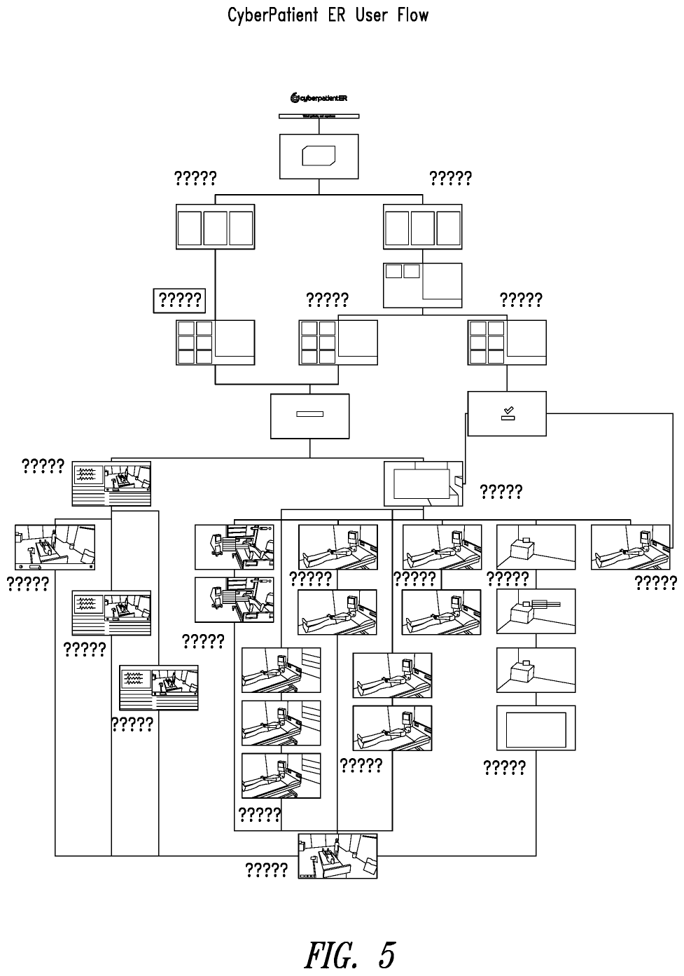

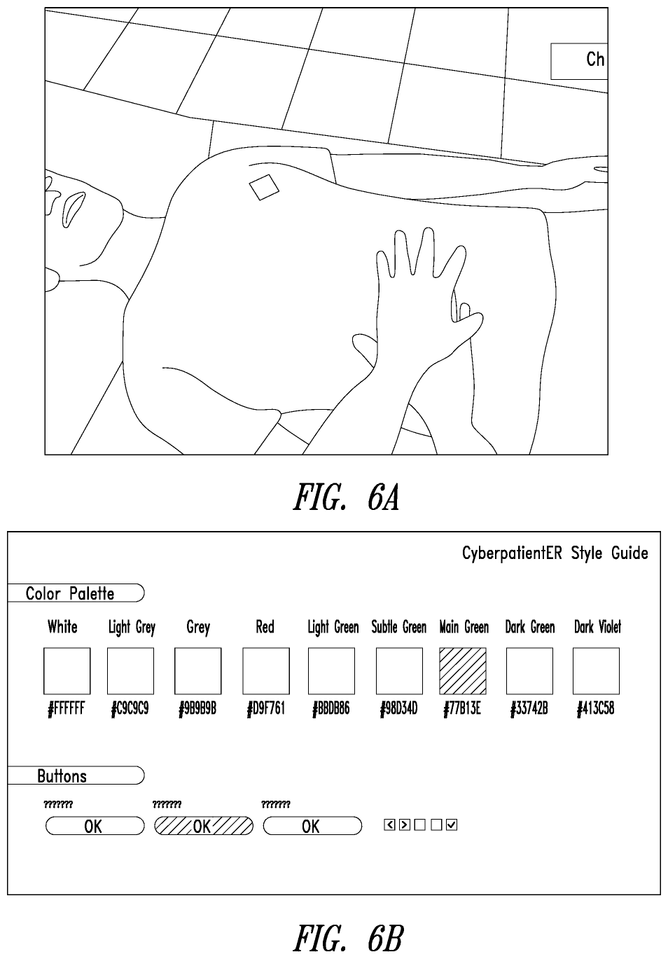

In an embodiment, the system implements a third-person point of view (POV). It was realized by the inventor that shifting from the third-person POV to the first-person POV might cause more complications than expected. While first-person POV as illustrated in FIG. 6A by way of example provides a better view on the patient and feels more realistic during interaction, no overview of the entire room or the Team can be easily provided (including name tags, speaking sign etc. of Team Members not in the Team Member's view). Furthermore, the first-person view creates user interface complications and takes more time to move around and interact, therefore influencing communications. First-person point of use also requires more complex controls and focuses the user on the animation—and instead of communication, which is the main focus. Therefore, in an embodiment, the system stays with a third-person POV.

FIG. 6B shows an illustrative user interface selection screen for selecting user interface parameters in accordance with an illustrative embodiment. Based on the style guide used in the Patient Avatar provided by the client, a sub-style guide was created to make sure the interface can fit for the 3D environment in the simulation. The substyle guide uses the same color palette and typography. On the basis of the original UI style guide, the Team added more elements into the guide. There are nine sections in the style guide: 1. Color palette; 2. Buttons; 3. Input box; 4. Dropdown menu; 5. Cards; 6. Others; 7. Icon; 8. Typography; 9. Logo.

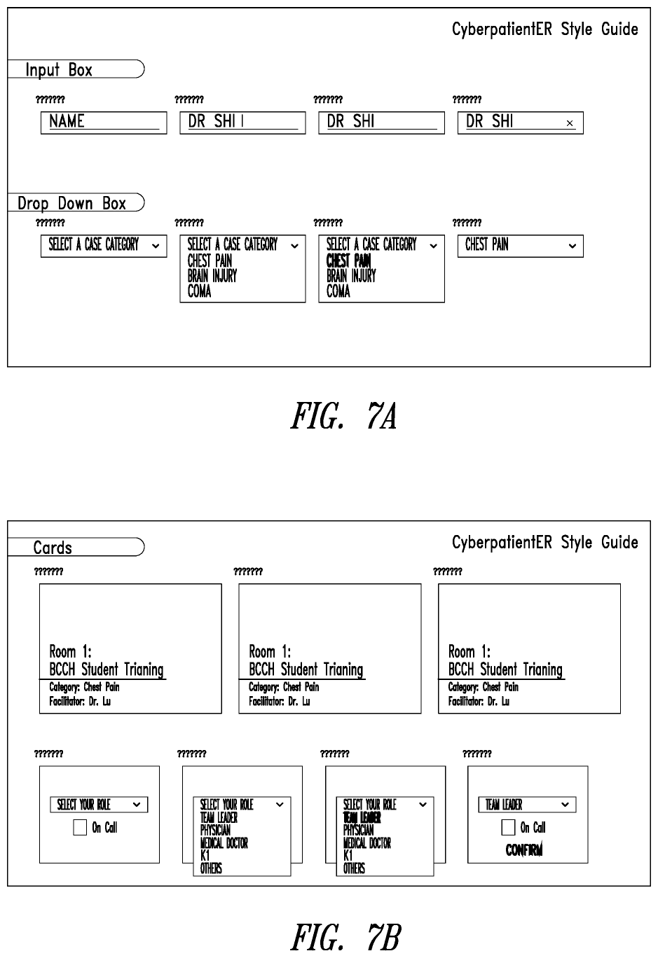

Now referring to FIGS. 7A-9A, shown are illustrative user interface screens of the present system in accordance with illustrative embodiments.

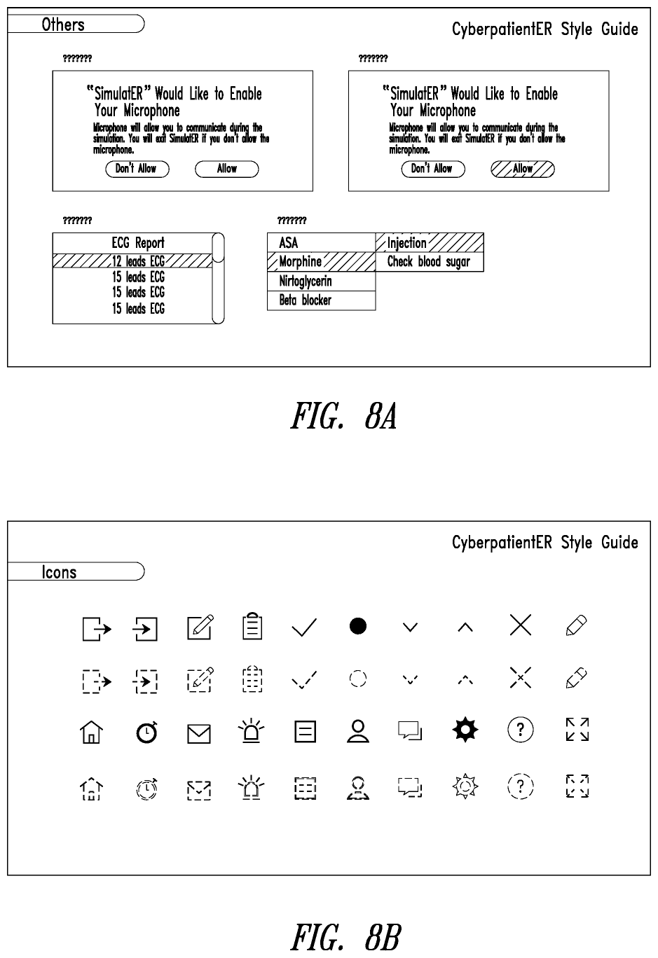

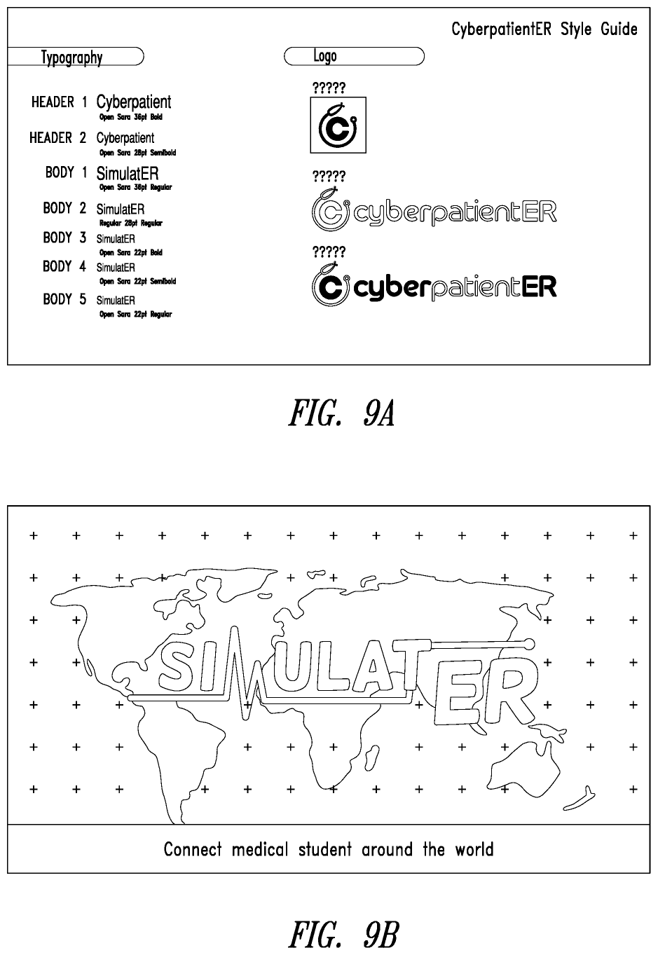

Now referring to FIGS. 9B and 10A, shown is an illustrative title page and a splash page of the system in accordance with an embodiment. In an embodiment, the system includes a 3D look and feel by adding opacity, gradient, and shadow.

FIG. 10B shows an illustrative login page in accordance with an embodiment. In an embodiment, simply typing in the name and choosing a category (facilitator or participant), the user can obtain easy access to the room selection page.

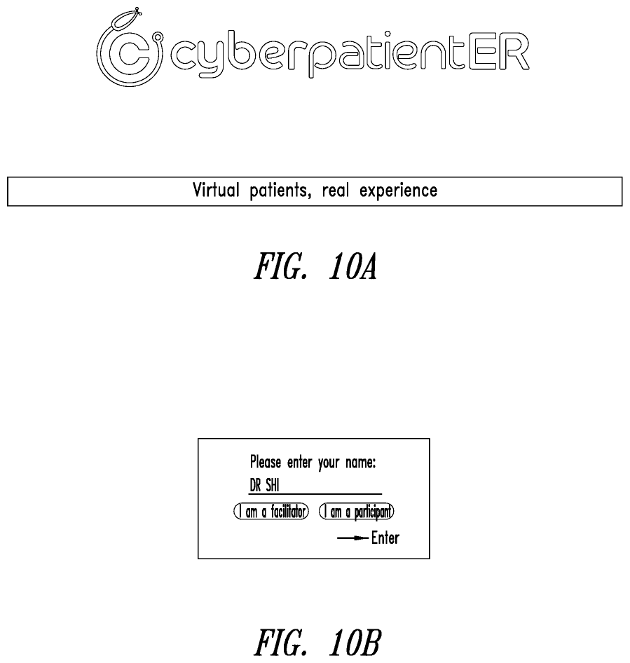

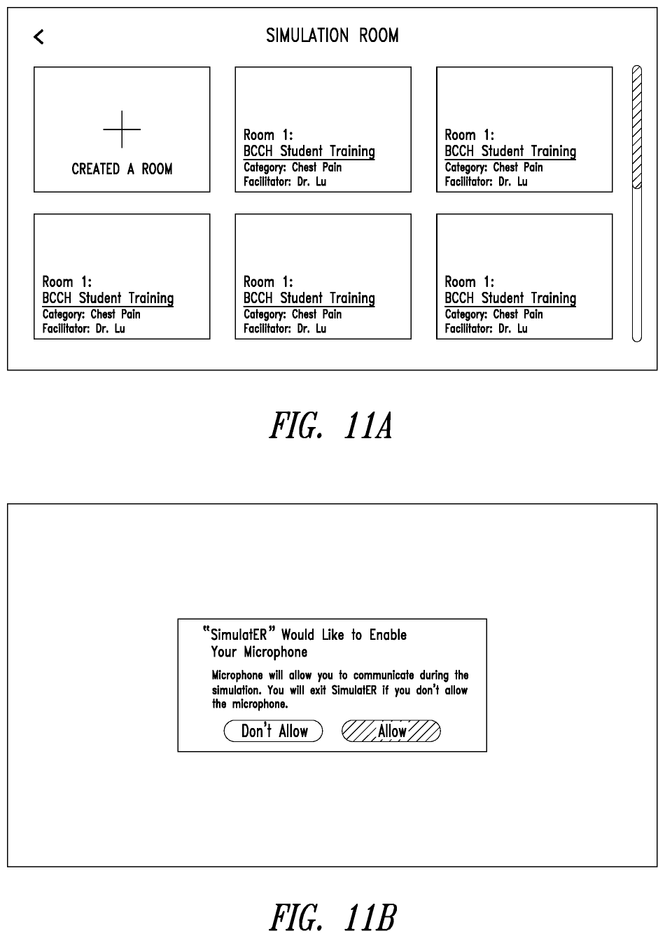

Now referring to FIG. 11A, shown is a room selection pages. Once the user enters the room selection page, he/she can see the room name, which category the scenario belongs to and who is the facilitator that leads the simulation. Users can get into the room based on their own Team's discussion before entering the simulation.

FIG. 11B shows an illustrative pop-up notification. This pop-up notification shows which users need to activate their microphone to proceed to the continuing process.

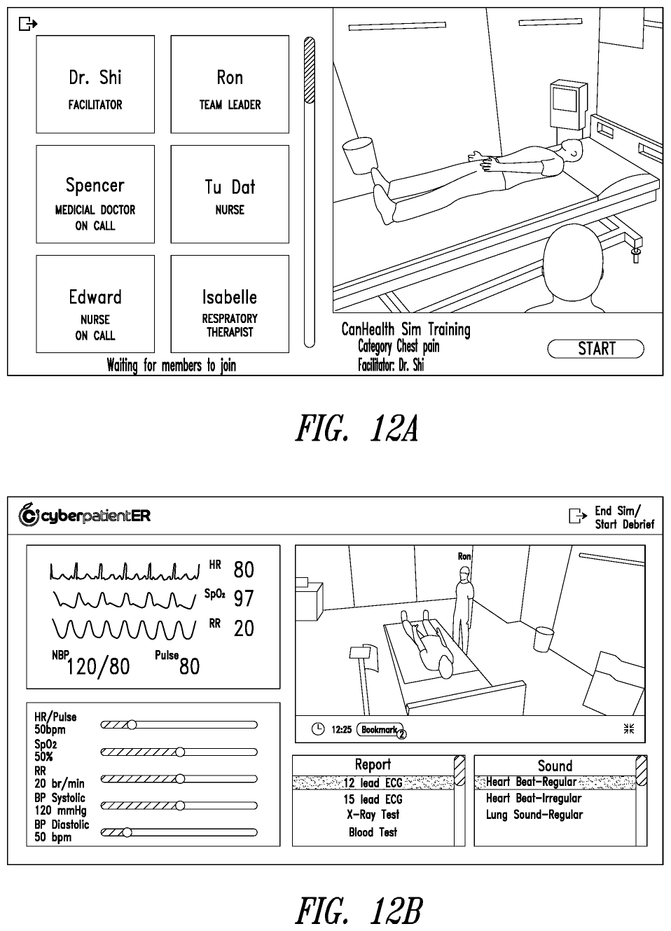

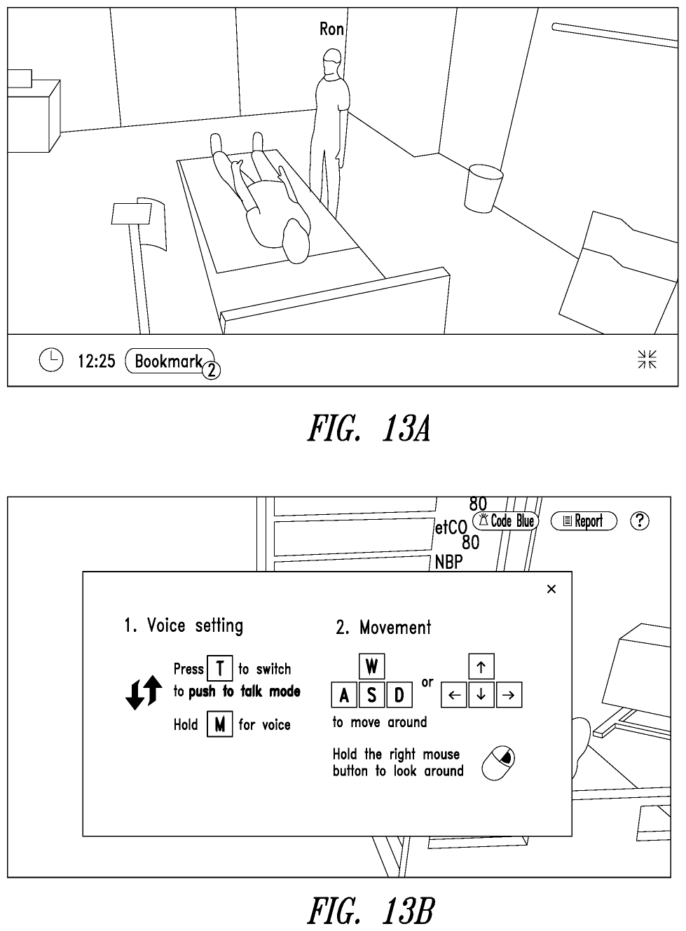

Now referring to FIG. 12A, shown is role selection page in accordance with an illustrative embodiment. In the role selection page, all of the users' microphones are activated and the facilitator can assign roles to each Member vocally. Each Team Member can choose the role based on what the facilitator said.

In an embodiment, there is a closed-loop communication video. When all Team Members enter the role selection room, under the guidance of the facilitator, they will watch a tutorial together. After that, they will choose their role based on the Team vocal discussion.

FIG. 12B shows a simulation screen of a Patient Avatar in accordance with an illustrative embodiment. As shown, there is a dashboard-style look and feels which contains several controller panels: Vital signs panel (it looks exactly the same as the screen of the ECG machine in the simulation); Parameter controller panel; live stream monitor; report and sound panel. For the live stream panel, the user has the ability to full screen the panel to have a clear view and observation of the ER simulation room.

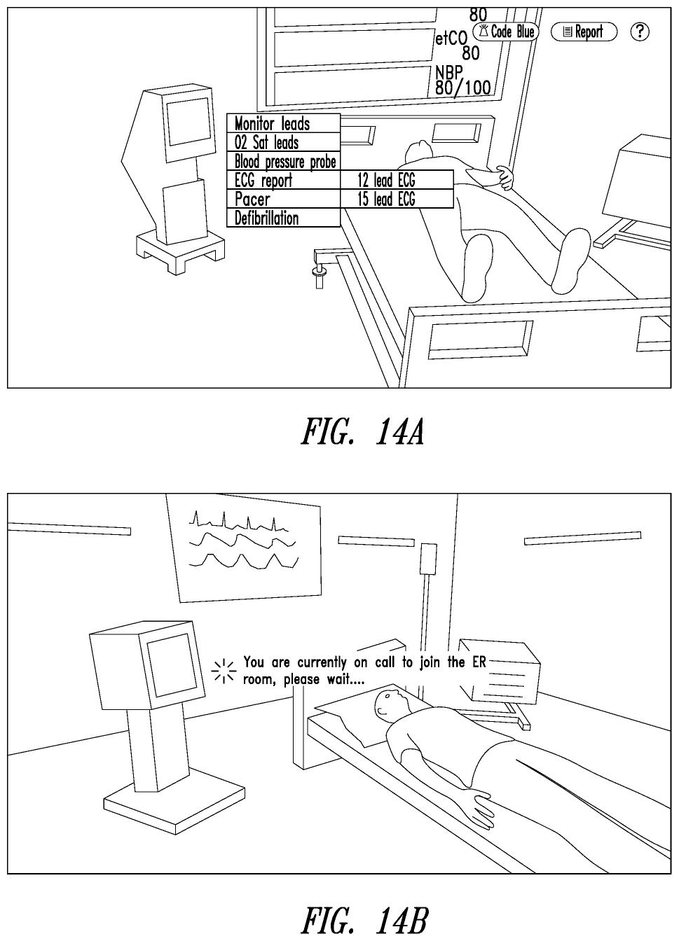

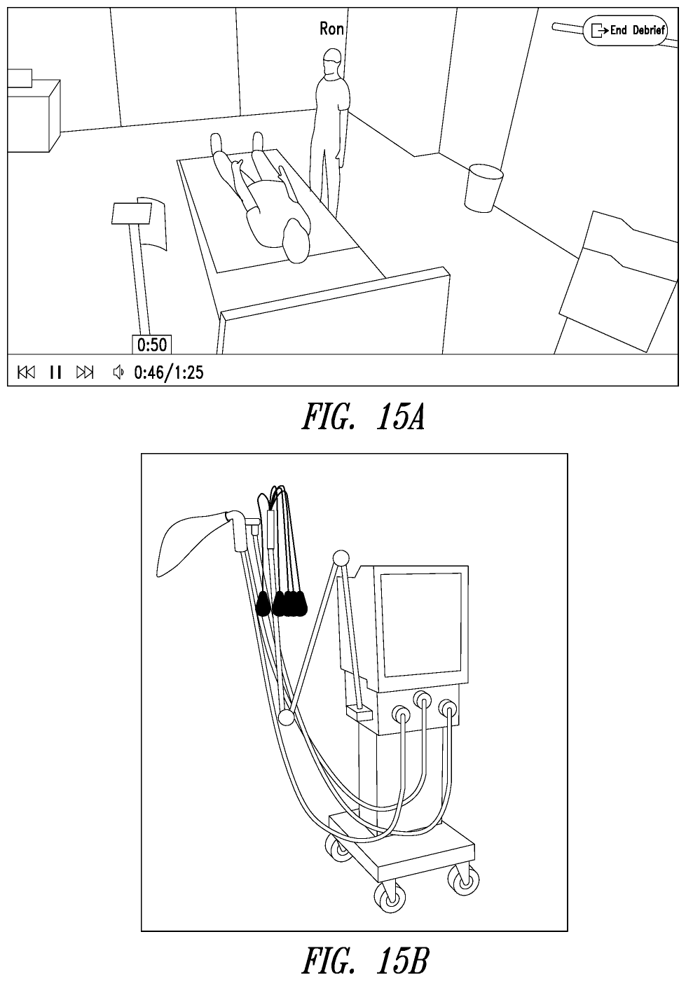

FIG. 13A a simulation screen with a participating Team Member in accordance with an illustrative embodiment.

FIG. 13B shows an on boarding pop-up page for a participating Team Member in accordance with an illustrative embodiment. The instructions explain how to control the avatar and communicate with each other.

Now referring to FIG. 13C, shown is a hidden panel which allows a user to hover-over an asset she/he wants to interact with. The object will be highlighted and as the user clicks on the asset, the semi-transparent panel shows up.

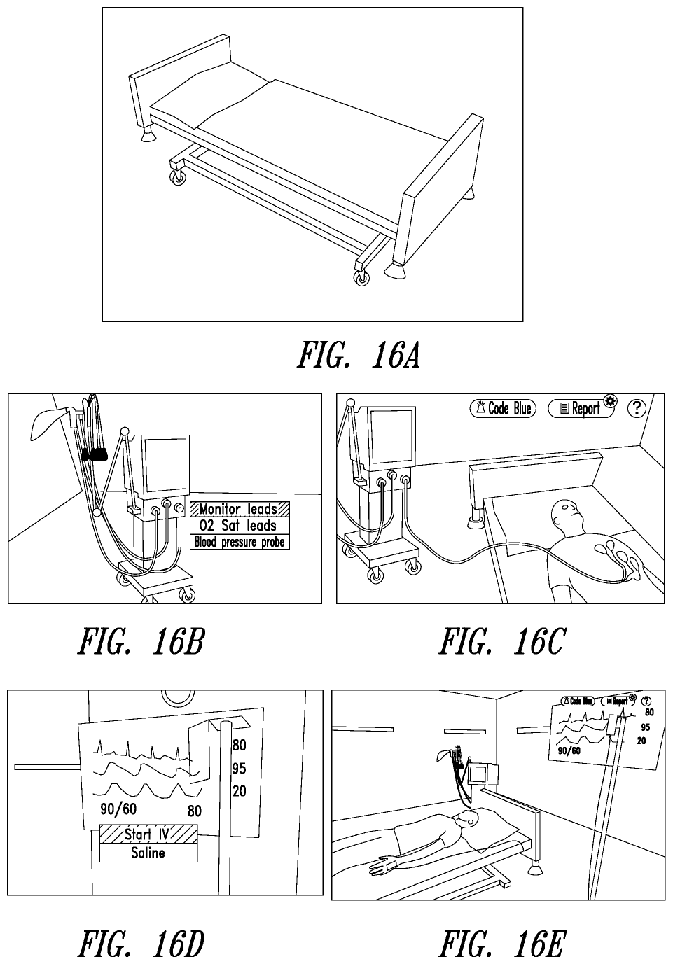

FIGS. 14A and 14B show an on-call waiting and debriefing room in accordance with an illustrative embodiment. After the Team finishes the simulation, they will go to the debrief session together with the facilitator. Please be mindful that for this part, all the assets can be found in the assets folder, as it hasn't been implemented into the build yet. Theoretically, in the debrief screen, there will be a full-screen video displayed which is a recording back in the simulation. There will be several spots highlighted in the timeline, which shows the bookmark the facilitator booked before. Based on the bookmark, the Team Member can jump to the exact time to discuss issues or things to work on that the facilitator has flagged.

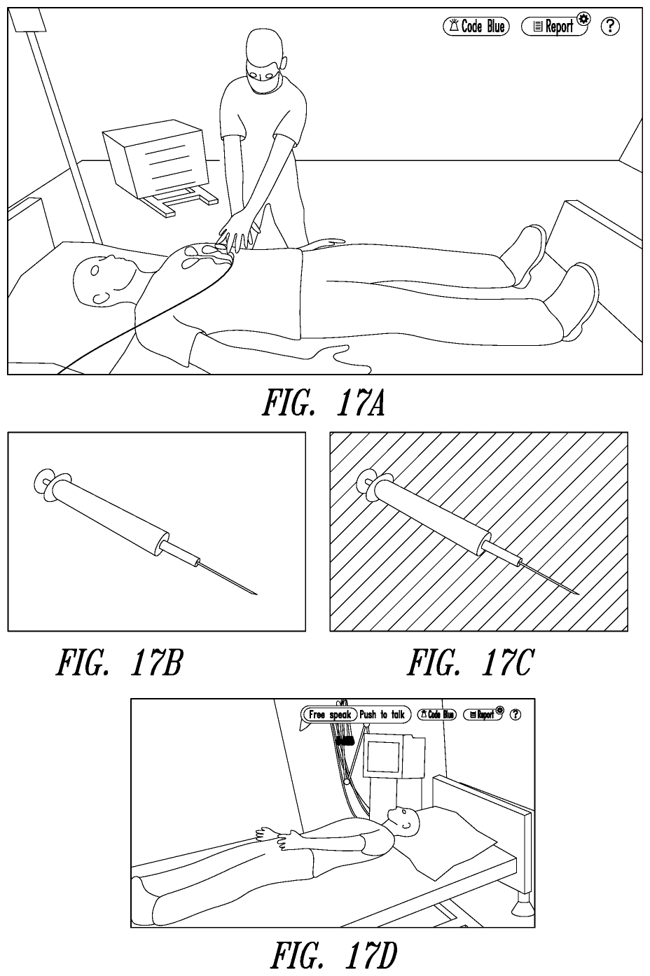

Now referring to FIGS. 15A-17D, shown are illustrative simulation screens with various objects with which participating Team Members may interact. As shown, the system utilizes illustrative 3-D objects to create a realistic simulation. For example, the simulation includes modular buildings, props, and characters. The objects may further include: ECG machine, the ER bed, the virtual patient and the TV Test Other than modifying several assets, the Team also created new interactive objects based on the actions required. By way of example, FIG. 15B shows an ECG machine: added one more “lead head” and created a major lead which split into five ECG leads. FIG. 16A illustrates an ER bed with which a user may interact. For example, the user can shrink the size, removed the bed handles and several small parts, etc.

FIGS. 16B to 16E illustrate additional objects and a Patient Avatar with which a user may interact.

In an embodiment, the system may be separated into multiple screens so that the system can enable the ability to highlight two different parts of a patient. The system also incorporates various device screens in a separate pop-up window to allow information to be shown clearly.

In an embodiment, the system may incorporate animations to demonstrate a procedure. For example, FIG. 17A shows a chest compression animation which allows a demonstration of the procedure within the simulated environment.

Based on a scenario that a teaching module requires, there are several actions that require additional interactive objects. Overall, there are multiple cut-scene actions, one animation, and one voice chat signifier created. Each object such as a syringe (FIGS. 17B and 17C) and an ECG machine (FIG. 17D) has controls with which Team Members can interact.

In an embodiment, a voice chat signifier is designed for the user to manually toggle between two voice chat modes: free speaking mode and press to talk mode. Users can use it in two different ways: 1. manually control the mode from the signifier on the screen (users can simply click on the button to change the mode). 2. press the hotkeys on the keyboard to change the mode. In this case, the user can see the voice chat signifier automatically change after the user toggles between the modes using the hotkeys.

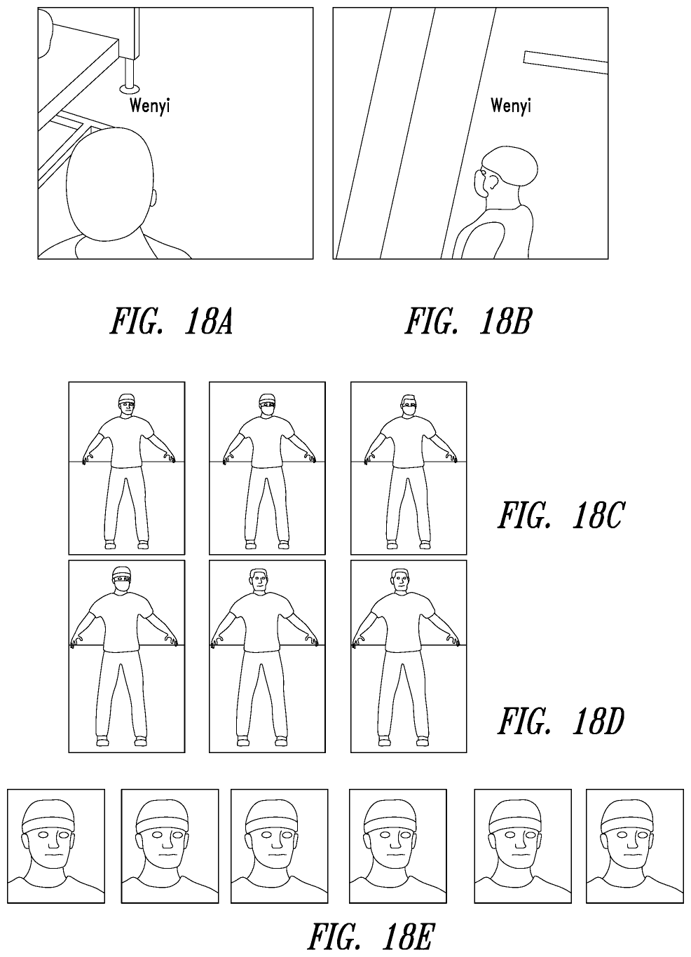

Now referring to FIGS. 18A-18E, shown are illustrative user interface screens for selecting and customizing participating Team Member Avatars in accordance with an illustrative embodiment.

Ideally, once Members enter the virtual emergency room, there will be name tags hovering above avatars, showing the name of each user. The default color of name tags is blue, in which the color will then turn red if a user starts talking. This function allows other users and the facilitator to see who are in the emergency room and who are using the voice chat function.

Users gain a sense of existence by having their avatars in the virtual emergency room. There are six initial avatars in the unity assets, three health professionals (FIG. 18C) and three patients (FIG. 18D). Diverse female and male doctor/Patient Avatars can be created by modifying the original avatars' attributes, such as faces, skin tones, hats, masks, and clothes.

As shown in FIG. 18E, by customizing avatars' attributes, many different variations of avatars can be created. Here is an example of customizing the face to show the possibility of customizing avatars.

### Navigation

With respect to navigation of Team Member Avatars, existing navigation solutions may be utilized. For example, the current navigation scheme of the application is based on Unity's provided Third Person User Control script. This control scheme was largely chosen due to the potential audience of the application. As users of the application may not have extensive experience in FPS (First person shooter) video games, the system utilizes a control scheme that is not deeply reliant on such experience.

Due to the point of view of the user's camera, a user's peripheral vision might be restricted. Therefore, a view panning feature is introduced to allow a user to pan the camera horizontally and vertically. However, because this panning of the camera does not move the character itself, the system allows the camera snap back to the character's facing direction to avoid confusion of having the user move the avatar forward while facing in a different direction. It was further suggested by the client to have the angle of the camera to retain the original angle to view vital signs on the TV screen. It is important to note that this concept of camera panning is strongly rooted in advanced First-Person Shooter simulators such as ARMA.

A mobile version of the simulation could be another option for a first-person Team Member simulation. It would make sense to enable the gyroscope on the phone so that the user would be able to tilt the phone in whatever direction to change perspective. This could be more intuitive as compared to the variety of controls currently enabled on our laptop platform.

In an embodiment, Unity may be used as the game engine for this application due to the versatility and in-depth documentation. The game engine not only provides the 3D environment and cameras to produce a first-person view simulation for Team Members, but Unity also provides a marketplace from which assets and tooling can be imported into the application. In addition, Unity allows the development Team to quickly produce multi-platform builds for testing and production.

In development of the application, the inventor targeted and tested the simulator with Windows and Mac computers. Due to the in-depth documentation and community support for Unity, it is believed that the engine is suitable for this application and is beginner-friendly for those who do not have 3D engine experience.

### Patient Avatar

Because the given data (data points) for the calculation of the new patient's vital signs were too simplified, the following assumptions were made to serve the purpose of the proof of the concept:
Only consider the difference in vital sign values based on the applied dose
The change of vital signs is linear
Medication has an effective period/decay (default 300 s). If the same medication is applied within this time period, the dosage will accumulate. For example, if a medication profile has the following: 0.5 mg will increase HR by 10, 1 mg will increase HR by 30. If a 0.5 mg is applied the HR will increase from 60 to 70 bpm (60+10) and if within 300s of this medication another 0.5 mg is applied, the HR will increase from 70 to 90 (60+30).
Within the effective period of a medication, the max change is the max difference between the first and last data points
The challenge was to have generic methods and data structures that allowed new medications to be added easily. The designed algorithm was straight forward as below:
if inject a medication: if the SAME medication is applied: if the current time is still in the effective period* of the last dose:
accumulate dose
else use the new dose
calculate the new vital signs from the data points save the medication history
save the patient vital sign history

The smart mannequin build was tested and showed the vital signs were changed correctly upon the applied medication.

Advantageously, the present system and method optimizes Team training and soft skill competencies. It can also be used in other areas of endeavor wherever Team training and soft skills such as communication skills are needed. While a simplified version of a Patient Avatar may serve the purpose of facilitating team communication, more sophisticated Patient Avatars are needed for facilitating more advanced medical scenarios. For example, a Patient Avatar with normal physiological parameters that will react dose dependently on drugs and environmental changes such as oxygen level, heat and parameters may be utilized. Such a Patient Avatar would be as close to a real human being as possible. For the realization of this goal, AI and machine learning capabilities may be utilized to iteratively improve the Patient Avatar algorithms to provide a realistic, sophisticated Patient Avatar platform.

### Haptic Feedback

In an embodiment, the present team training system may incorporate haptic feedback technology.

For example, the Patient Avatar's skin surface, and/or surface of internal organs, can be coded mathematically to display feedback to a haptic interface. Team Members may each wear a haptic glove or instruments connected to the haptic interface that is/are controllable by Team Members through their Team Member Avatars. In this instance, Team Members will be able to feel the interaction with a Patient Avatar through their Team Member Avatar. For example, a Team Member may perform palpation, percussion, auscultation, as well as manipulation such as intubation with tactile sensation. This will make the environment very realistic, close to a direct contact with an actual patient. This will also provide the environment for Team Members to perform physical examination, surgical procedures and/or other invasive or noninvasive manipulations with the use of this technology inside the ER or in separate instances.

In an embodiment, each Team Member Avatar's interaction with a Patient Avatar may be simultaneously processed such that the Patient Avatar responds in real-time to multiple simultaneous inputs from the Team Members. Thus, the Patient Avatar provides a realistic physiological response and may provide a haptic feedback to one or more Team Members interacting with the Patient Avatar. Therefore, apart from interacting via voice chat and visually via a computer screen or display, each Team Member may also interact with each other through haptic feedback via the Patient Avatar.

Apart from interacting via the Patient Avatar, each Team Member may also interact with various objects such as tools or devices within the virtual environment via their Team Member Avatar. Again, each Team Member may interact with another Team Member via the objects, for example by passing a tool between Team Members, or by assisting another Team Member to monitor a device and inform another Team Member of the status or readings on the device.

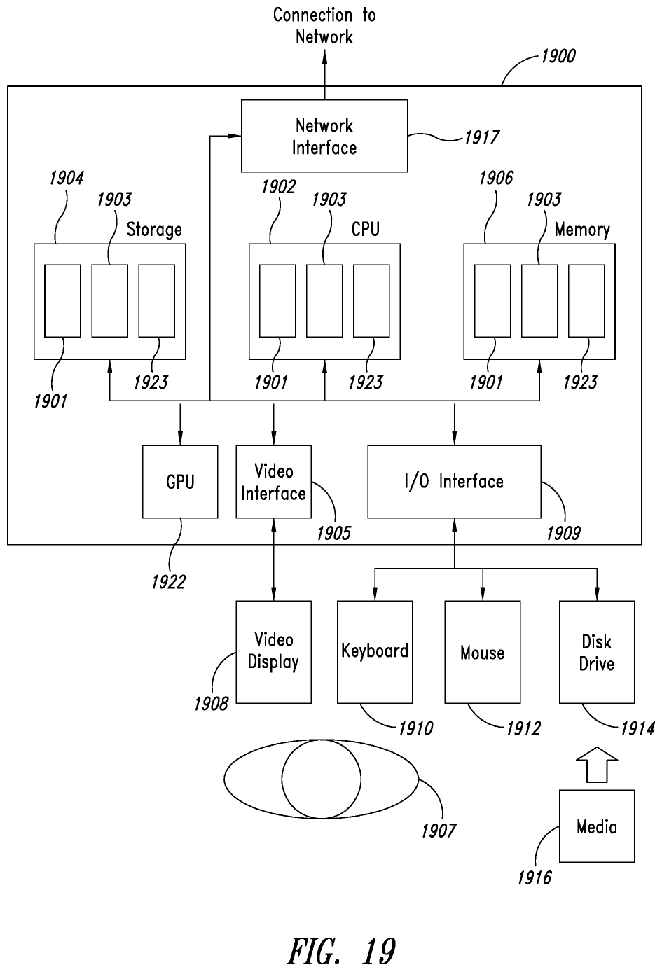

Now referring to FIG. 19 shown is a schematic block diagram of a generic computing device that may provide a suitable operating environment in one or more embodiments. A suitably configured computer device, and associated communications networks, devices, software and firmware may provide a platform for enabling one or more embodiments as described above. By way of example, FIG. 19 shows a generic computer device 1900 that may include a central processing unit (“CPU”) 1902 connected to a storage unit 1904 and to a random access memory 1906. The CPU 1902 may process an operating system 1901, application program 1903, and data 1923. The operating system 1901, application program 1903, and data 1923 may be stored in storage unit 1904 and loaded into memory 106, as may be required1. Computer device 1100 may further include a graphics processing unit (GPU) 1922 which is operatively connected to CPU 1902 and to memory 1906 to offload intensive image processing calculations from CPU 1902 and run these calculations in parallel with CPU 1902. An operator 1910 may interact with the computer device 1900 using a video display 1908 connected by a video interface 1905, and various input/output devices such as a keyboard 1910, pointer 1912, and storage 1914 connected by an I/O interface 1909. In known manner, the pointer 1912 may be configured to control movement of a cursor or pointer icon in the video display 1908, and to operate various graphical user interface (GUI) controls appearing in the video display 1908. The computer device 1900 may form part of a network via a network interface 1911, allowing the computer device 1900 to communicate with other suitably configured data processing systems or circuits. A non-transitory medium 1916 may be used to store executable code embodying one or more embodiments of the present method on the generic computing device 1900.

Advantageously, by providing simultaneous interaction between the Team Member Avatars and the Patient Avatar, and with various tools or devices within the virtual environment, the present system facilitates teamwork and communication between Team Members as they cooperate in treating the Patient Avatar.

It should be noted that automatically created Patient Avatars with haptic user interfaces can be used in any other instances related to training of health professionals as well as healthcare events and instances.

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementation of the various embodiments described herein.

Thus, in an aspect, there is provided a computer-implement system for virtual team training and assessment of soft skill competencies in a hospital environment, the system including a plurality of networked computers, each computer including a processor, memory, and storage, and adapted to provide: a virtual simulated hospital environment having a plurality of simulated objects; a virtual patient avatar configured to provide various team training scenarios; and a user interface for each team member controlling a team member avatar to interact with the patient avatar and the plurality of simulated objects to perform one or more team training scenarios within the virtual simulated hospital environment.

In an embodiment, the system is further configured to process simultaneous inputs from team members interacting with the virtual patient avatar, and output a realistic physiological response to the simultaneous inputs.

In another embodiment, the team training is conducted through one or more of audio and video interaction and animation between the team members, and with one or more instructors.

In another embodiment, the team training is conducted through one or more of haptic feedback interactions between the team members and the patient avatar.

In another embodiment, the team training is conducted through one or more of haptic feedback interactions between the team members and one or more objects within the virtual simulated hospital environment.

In another embodiment, the patient avatar is controlled by one or more Instructors to modify the training scenario in real-time.

In another embodiment, the patient avatar is controlled by an artificial intelligence engine to simulate a training scenario and to modify the training scenario in real-time.

In another aspect, there is provided a computer-implemented method of virtual team training and assessment of soft skill competencies in a hospital environment, the method implemented on a system including a plurality of networked computers, each computer including a processor, memory, and storage, and comprising: providing a virtual simulated hospital environment having a plurality of simulated objects; providing a virtual patient avatar configured to provide various team training scenarios; and providing a user interface for each team member controlling a team member avatar to interact with the patient avatar and the plurality of simulated objects to perform one or more team training scenarios within the virtual simulated hospital environment.

In an embodiment, the computer-implemented method further comprises processing simultaneous inputs from team members interacting with the virtual patient avatar, and outputting a realistic physiological response to the simultaneous inputs.

In another embodiment, the computer-implemented method further comprises conducting the team training through one or more of audio and video interaction and animation between the team members, and with one or more instructors.

In another embodiment, the computer-implemented method further comprises conducting the team training through one or more of haptic feedback interactions between the team members and the patient avatar.

In another embodiment, the computer-implemented method further comprises conducting the team training through one or more of haptic feedback interactions between the team members and one or more objects within the virtual simulated hospital environment.

In another embodiment, the computer-implemented method further comprises controlling the patient avatar by one or more instructors to modify the training scenario in real-time.

In another embodiment, the computer-implemented method further comprises controlling the patient avatar by an artificial intelligence engine to simulate a training scenario and to modify the training scenario in real-time.

In another aspect, there is provide a non-transitory computer readable media containing computer executable code for performing a method of virtual team training and assessment of soft skill competencies in a hospital environment, comprising: code for providing a virtual simulated hospital environment having a plurality of simulated objects; code for providing a virtual patient avatar configured to provide various team training scenarios; and code for providing a user interface for each team member controlling a team member avatar to interact with the patient avatar and the plurality of simulated objects to perform one or more team training scenarios within the virtual simulated hospital environment.

In an embodiment, the non-transitory computer readable media further comprises code for processing simultaneous inputs from team members interacting with the virtual patient avatar, and outputting a realistic physiological response to the simultaneous inputs.

In another embodiment, the non-transitory computer readable media further comprises code for conducting the team training through one or more of audio and video interaction and animation between the team members, and with one or more instructors.

In another embodiment, the non-transitory computer readable media further comprises code for conducting the team training through one or more of haptic feedback interactions between the team members and the patient avatar.

In another embodiment, the non-transitory computer readable media further comprises code for conducting the team training through one or more of haptic feedback interactions between the team members and one or more objects within the virtual simulated hospital environment.

In another embodiment, the non-transitory computer readable media further comprises code for controlling the patient avatar by one or more instructors to modify the training scenario in real-time.

While illustrative embodiments have been described above by way of example, it will be appreciated that various changes and modifications may be made without departing from the scope of the invention, which is defined by the following claims.

1. A computer-implement system for virtual team training and assessment of soft skill competencies in a hospital environment, the system including a plurality of networked computers, each computer including a processor, memory, and storage, and adapted to provide:
a virtual simulated hospital environment having a plurality of simulated objects;
a virtual patient avatar configured to provide various team training scenarios; and
a user interface for each team member controlling a team member avatar to interact with the patient avatar and the plurality of simulated objects to perform one or more team training scenarios within the virtual simulated hospital environment.

2. The computer-implemented system of claim 1, wherein the system is further configured to process simultaneous inputs from team members interacting with the virtual patient avatar, and output a realistic physiological response to the simultaneous inputs.

3. The computer-implemented system of claim 1, wherein the team training is conducted through one or more of audio and video interaction and animation between the team members, and with one or more instructors.

4. The computer-implemented system of claim 1, wherein the team training is conducted through one or more of haptic feedback interactions between the team members and the patient avatar.

5. The computer-implemented system of claim 1, wherein the team training is conducted through one or more of haptic feedback interactions between the team members and one or more objects within the virtual simulated hospital environment.

6. The computer-implemented system of claim 1, wherein the patient avatar is controlled by one or more Instructors to modify the training scenario in real-time.

7. The computer-implemented system of claim 1, wherein the patient avatar is controlled by an artificial intelligence engine to simulate a training scenario and to modify the training scenario in real-time.

8. A computer-implemented method of virtual team training and assessment of soft skill competencies in a hospital environment, the method implemented on a system including a plurality of networked computers, each computer including a processor, memory, and storage, and comprising:
providing a virtual simulated hospital environment having a plurality of simulated objects;
providing a virtual patient avatar configured to provide various team training scenarios; and
providing a user interface for each team member controlling a team member avatar to interact with the patient avatar and the plurality of simulated objects to perform one or more team training scenarios within the virtual simulated hospital environment.

9. The computer-implemented method of claim 8, further comprising processing simultaneous inputs from team members interacting with the virtual patient avatar, and outputting a realistic physiological response to the simultaneous inputs.

10. The computer-implemented method of claim 8, further comprising conducting the team training through one or more of audio and video interaction and animation between the team members, and with one or more instructors.

11. The computer-implemented method of claim 8, further comprising conducting the team training through one or more of haptic feedback interactions between the team members and the patient avatar.

12. The computer-implemented method of claim 8, further comprising conducting the team training through one or more of haptic feedback interactions between the team members and one or more objects within the virtual simulated hospital environment.

13. The computer-implemented method of claim 8, further comprising controlling the patient avatar by one or more instructors to modify the training scenario in real-time.

14. The computer-implemented method of claim 8, further comprising controlling the patient avatar by an artificial intelligence engine to simulate a training scenario and to modify the training scenario in real-time.

15. A non-transitory computer readable media containing computer executable code for performing a method of virtual team training and assessment of soft skill competencies in a hospital environment, comprising:
code for providing a virtual simulated hospital environment having a plurality of simulated objects;
code for providing a virtual patient avatar configured to provide various team training scenarios; and
code for providing a user interface for each team member controlling a team member avatar to interact with the patient avatar and the plurality of simulated objects to perform one or more team training scenarios within the virtual simulated hospital environment.

16. The non-transitory computer readable media of claim 15, further comprising code for processing simultaneous inputs from team members interacting with the virtual patient avatar, and outputting a realistic physiological response to the simultaneous inputs.

17. The non-transitory computer readable media of claim 15, further comprising code for conducting the team training through one or more of audio and video interaction and animation between the team members, and with one or more instructors.

18. The non-transitory computer readable media of claim 5, further comprising code for conducting the team training through one or more of haptic feedback interactions between the team members and the patient avatar.

19. The non-transitory computer readable media of claim 15, further comprising code for conducting the team training through one or more of haptic feedback interactions between the team members and one or more objects within the virtual simulated hospital environment.

20. The non-transitory computer readable media of claim 5, further comprising code for controlling the patient avatar by one or more instructors to modify the training scenario in real-time.
